(19) United States
(12) Patent Application Publication
(10) Pub. No.: US 2025/0241868 A1
(43) Pub. Date: Jul. 31, 2025

(54) METHODS OF TREATING CANCER USING THIOREDOXIN REDUCTASE INHIBITORS

(71) Applicant: Father Flanagan's Boys' Home Doing Business as Boys Town National Research Hospital, Omaha, NE (US)

(72) Inventor: M. Rohan Fernando, Omaha, NE (US)

(21) Appl. No.: 19/038,111

(22) Filed: Jan. 27, 2025

Related U.S. Application Data
(60) Provisional application No. 63/625,567, filed on Jan. 26, 2024.

(51) Int. Cl. A61K 31/047; A61K 31/341; A61K 38/05

(52) U.S. Cl. CPC A61K 31/047; A61K 31/341; A61K 38/05

(57) ABSTRACT

The effects of compositions 2-bromo-2-nitro-1,3-propanediol are discussed herein, including an anticancer effect of the compositions. Methods related to treating cancer in a subject comprising administering compositions comprising 2-bromo-2-nitro-1,3-propanediol and methods related to inhibiting the activity of thioredoxin reductase are also provided. Dosage ranges are disclosed along with compatible treatment regimens.

### CROSS-REFERENCE TO RELATED APPLICATIONS

This application claims priority to U.S. Provisional Patent Application No. 63/625,567, filed on Jan. 26, 2024. The Provisional application is herein incorporated by reference in its entirety, including without limitation, the specification, claims, and abstract, as well as any figures, tables, appendices, or drawings thereof.

### TECHNICAL FIELD

The present disclosure relates generally to anticancer therapies. More particularly, the disclosure relates to thioredoxin reductase inhibitors and their use in the treatment of cancer.

### BACKGROUND

Cancer treatment remains one of the biggest unmet medical needs. While there have been advances in cancer therapy during the last decades, cancer remains a leading cause of death. Thus, the demand for new cancer therapies is ever increasing.

The mammalian thioredoxin system (TRXS) encompasses the interplay of several essential components, namely thioredoxin reductase (TR), thioredoxin (TRX), and NADPH. Elevated levels of TRX and TR have been observed in several types of cancers. Furthermore, for some cancers, elevated levels can indicate poor prognosis. Elevated levels of TRXS can disrupt cell cycle regulation through excessive activation of growth factors and inhibition of apoptosis, ultimately leading to the malignant transformation of cells.

Thus, there is a need for new cancer therapies capable of inhibiting the activity of TRX and TR.

### SUMMARY

Methods of treating cancer in a subject in need thereof are provided. In some embodiments, the methods comprise administering to the subject a composition comprising a therapeutically effective amount of 2-bromo-2-nitro-1,3-propanediol.

Methods of inhibiting the activity of thioredoxin reductase in a cell of a subject are provided. In some embodiments, the methods comprise administering to the subject a composition comprising a therapeutically effective amount of 2-bromo-2-nitro-1,3-propanediol. In some embodiment, the subject has cancer.

### BRIEF DESCRIPTION OF THE DRAWINGS

FIGS. 1A-IC show the effect of 2-bromo-2-nitro-1,3-propanediol (BP) on human recombinant TR (1A) and HeLa cell lysate (1B) TR activities. TR activity of human recombinant enzyme or Hela cell lysate was determined in the presence or absence of BP. TR activity was expressed as the percentage of those of the untreated enzyme or cell lysate. BP concentrations used in the reaction mixture were 12.5, 25, and 50 μM. The IC50 value for human recombinant TR was 21 μM. For HeLa cell lysate IC50 was 20.5 μM. 1C shows the reversible nature of BP mediated TR inhibition in human recombinant TR activity. TR was inactivated and then treated with dithiothreitol (DTT) as described in Example 1. Excess DTT was removed using a Sephadex™ G-25 column and TR activity was measured as described. The results are based on three independent experiments. Error bars indicates STD.

FIGS. 2A-2B show the effect of BP on HeLa cell viability (2A) and c-fos mRNA expression (2B). 2A, HeLa cells were treated with 12.5, 25, 50 and 100 μM BP for 24 hours and cell viability assessed as described in Examples 1 and 2. A dose dependent decrease in cell viability was observed and LD50 value for HeLa cells was 21 μM. 2B, HeLa cells were either treated or untreated with 50 μM BP for 4 or 24 hours. After indicated incubation periods cells were used to extract RNA and measure c-fos mRNA level using droplet digital PCR technology as described in Examples 1 and 2. Error bars indicates STD. * P<0.004.

FIGS. 3A-3B show the effect of BP on HeLa cell apoptosis. 3A, untreated Hela cells stained with Hoechst 33342 nuclear stain. 3B, HeLa cells treated with 50 μM BP for 24 hours and stained with Hoechst 33342 staining showing typical apoptotic nuclear morphological changes. The arrowheads indicate apoptotic cells, which exhibited highly condensed and fragmented nuclear morphologies.

### DETAILED DESCRIPTION

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments without undue experimentation, but the preferred materials and methods are described herein. In describing and claiming the embodiments, the following terminology will be used in accordance with the definitions set out below.

It is further to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾%. This applies regardless of the breadth of the range.

As used herein, the term “and/or”, e.g., “X and/or Y” shall be understood to mean either “X and Y” or “X or Y” and shall be taken to provide explicit support for both meanings or for either meaning, e.g. A and/or B includes the options i) A, ii) B or iii) A and B.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

The methods and compositions of the present disclosure may comprise, consist essentially of, or consist of the components and ingredients of the present disclosure as well as other ingredients described herein. As used herein, “consisting essentially of” means that the methods, systems, apparatuses and compositions may include additional steps, components or ingredients, but only if the additional steps, components or ingredients do not materially alter the basic and novel characteristics of the claimed methods, systems, apparatuses, and compositions.

The term “about,” as used herein, refers to variation in the numerical quantity that can occur, for example, through typical measuring techniques and equipment, with respect to any quantifiable variable, including, but not limited to, cell population, cell viability, concentration, dosage, mass, pH, temperature, time, and volume. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

In the present disclosure, an “effective amount” or “therapeutically effective amount” of a compound or of a composition of the present disclosure is that amount of such compound and/or composition that is sufficient to effect beneficial or desired results as described herein. In terms of treatment of a mammal, e.g., a human patient, an “effective amount” is an amount sufficient to treat, reduce, manage, palliate, ameliorate, or stabilize a condition as compared to the absence of the compound or composition.

The terms “include” and “including” when used in reference to a list of materials refer to but are not limited to the materials so listed.

As used herein, “pharmaceutically acceptable” refers to compounds, materials, compositions, and/or dosage forms which are suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. A “pharmaceutically acceptable excipient” includes any and all carriers, solvents, growth media, dispersion media, coatings, adjuvants, fillers, buffers, stabilizers, lubricants, stabilizing agents, diluents, preservatives, inactivating agents, antimicrobial, antibacterial and antifungal agents, isotonic agents, adsorption delaying agents, and the like.

Such ingredients include those that are safe and appropriate for use in medical or pharmaceutical applications.

The term “subject” as used herein refers to any living being that would benefit from the compositions and methods described herein. For example, the subject may be an animal, including a human, avian, bovine, canine, equine, feline, hircine, lupine, murine, ovine, and porcine animal. Subjects may also be domesticated animals such as cats, dogs, rabbits, guinea pigs, ferrets, hamsters, mice, gerbils, horses, cows, goats, sheep, donkeys, pigs, and the like.

In certain embodiments, the subject is a human.

The methods and compositions may comprise, consist essentially of, or consist of the components and ingredients as well as other ingredients described herein. As used herein, “consisting essentially of” means that the methods and compositions may include additional steps, components or ingredients, but only if the additional steps, components or ingredients do not materially alter the basic and novel characteristics of the claimed methods and compositions.

The mammalian thioredoxin system (TRXS) encompasses the interplay of several essential components, namely thioredoxin reductase (TR), thioredoxin (TRX), and NADPH. TRX, a protein weighing approximately 12 kDa, possesses an active site with two redox-active cysteine residues. TR, on the other hand, is a selenoprotein with a molecular mass of approximately 55 kDa, characterized by an active site featuring a selenocysteine and a cysteine residue. The oxidation state of TRX is reduced by TR and NADPH. Ubiquitously expressed, TRXS plays a crucial role in maintaining cellular and extracellular redox homeostasis. One of its primary functions is to sustain a low redox potential and a high level of free thiols in cells, in conjunction with cellular glutathione (GSH). The reduction of oxidized GSH is facilitated by glutathione reductase and NADPH. Additionally, TRXS participates in DNA biosynthesis by serving as a hydrogen donor for the enzyme ribonucleotide reductase. Moreover, TRXS acts as a hydrogen donor for methionine sulfoxide reductase, an enzyme crucial for reducing methionine sulfoxide residues in proteins damaged by oxidation. In mammalian cells, TRXS assumes a pivotal role in the redox regulation of transcription factors such as NFkB and AP-1. Furthermore, TRXS plays a major role in the regeneration of oxidatively damaged proteins in mammalian cells.

Elevated levels of TRX and TR have been observed in several types of cancers. The tumor microenvironment, characterized by oxidative stress or hypoxia, can induce increased levels of TRX and TR in cancerous cells. The augmented expression of TRX and TR may contribute to cancer progression and drug resistance by suppressing oxidative stress and hypoxic conditions. For example, have reported elevated levels of TRX and TR in thyroid cancer and non-small cell lung cancer. It has been postulated that elevated levels of TRXS could disrupt cell cycle regulation through excessive activation of growth factors and inhibition of apoptosis, ultimately leading to the malignant transformation of cells. Consequently, thioredoxin reductase is a potential molecular target for cancer therapy.

BP is a broad-spectrum antimicrobial agent widely employed in various industries, including pharmaceutical and cosmetic sectors, as a preservative. This compound demonstrates high water solubility and exerts antimicrobial activity at low concentrations. BP possesses the ability to oxidize thiol groups in proteins. Mammalian TR and TRX possess critical thiol groups, both structurally and within their active sites, which are essential for their biological activity. Without being limited by theory, oxidation of these critical thiol groups renders TR and TRX biologically inactive.

Methods of treating cancer in a subject are provided. In certain embodiments, the method comprises administering to the subject a composition comprising a therapeutically effective amount of 2-bromo-2-nitro-1,3-propanediol (BP), also known as bronopol.

Methods of inhibiting the activity of thioredoxin reductase in a cell of a subject are also provided. In some embodiments, the method comprises administering to the subject a composition comprising 2-bromo-2-nitro-1,3-propanediol.

In certain embodiments, the subject has cancer. In certain embodiments, the cancer is any cancer wherein TR and/or TRX is overexpressed. In certain embodiments, the cancer is thyroid cancer, breast cancer, endometrial cancer, colorectal cancer, gastric cancer, lung cancer, brain cancer, hepatic cancer, ovarian cancer, choriocarcinoma, osteosarcoma, hematologic cancer, pancreatic cancer, urogenital cancer, and/or uterine cancer. In certain embodiments, the cancer is breast cancer or prostate cancer. In certain embodiments the cancer is metastatic cancer that has spread to other parts of the body.

Suitable routes of administration include, but are not limited to, oral, parenteral (e.g., intravenous, intraarterial, rectal, subcutaneous, intramuscular, intraorbital, intracapsular, intraspinal, intraperitoneal, or intrasternal), topical (transdermal, intranasal, ocular, buccal, and sublingual), intravesical, intrathecal, enteral, pulmonary, intralymphatic, intracavital, vaginal, transurethral, intradermal, aural, intramammary, orthotopic, intratracheal, intralesional, percutaneous, endoscopical, transmucosal, and intestinal administration.

In certain embodiments, the composition is administered orally, intravenously, or intramuscularly.

The size of the dose of each therapy which is required for the therapeutic or prophylactic treatment of a particular disease state will necessarily be varied depending on the host treated, the route of administration and the severity of the illness being treated. Accordingly, the optimum dosage may be determined by the practitioner who is treating any particular patient and taking into consideration various factors known to modify the action of drugs including severity and type of disease, body weight, sex, diet, time and route of administration, other medications and other relevant clinical factors. It may also be necessary or desirable to reduce the doses of the components of the combination treatments in order to reduce toxicity. Therapeutically effective dosages may be determined by either in vitro or in vivo methods.

In certain embodiments, an effective dose ranges from about 0.01 mg to about 10 g per square meter body area of the subject. In certain embodiments, the dose ranges from about 0.01 mg/kg to about 10 g/kg, from about 0.05 mg/kg to about 10 g/kg, from about 0.1 mg/kg to about 5 g/kg, from about 0.1 mg/kg to about 1 g/kg, from about 0.1 mg/kg to about 500 mg/kg, from about 0.1 mg/kg to about 100 mg/kg, from about 0.5 mg/kg to about 50 mg/kg, from about 0.5 mg/kg to about 25 mg/kg, from about 0.5 mg/kg to about 10 mg/kg, from about 1 mg/kg to about 5 g/kg, or any range therein. A unit dosage form such as a tablet, capsule, or bolus may contain, for example, about 1 mg to about 100 g of active ingredient depending on the subject.

The dosages and schedules may vary according to the particular disease state and the overall condition of the patient. Dosages and schedules may also vary if, in addition to administration of a composition of the present disclosure, one or more additional chemotherapeutic agents is/are used. Scheduling can be determined by the practitioner who is treating any particular patient.

In an embodiment, the treatment may be performed by administration of the novel compounds to the subject in need thereof or by administration directly to the site of the tumor or other cancer cells. The treatment may be performed in conjunction with administration of a chemotherapeutic agent or additional treatment agent (e.g., as part of a treatment regimen), either simultaneously, in a single composition or in separate compositions, or sequentially, with the novel compounds of the present disclosure. The treatment may be performed by administration of components in any order and in any combination. The treatment may also be performed using more than one chemotherapeutic agent, or other type of treatment. Further, the treatment may be performed by providing multiple administrations of the compositions. One skilled in the art will ascertain these variations in treatment regimens employing the novel compounds disclosed herein.

The compounds or pharmaceutical compositions of the disclosure can be used in combination with at least one or more chemotherapeutic agents. In some embodiments, the chemotherapeutic may comprise mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. Non-limiting examples are chemotherapeutic agents, cytotoxic agents, and non-peptide small molecules such as Gleevec® (Imatinib Mesylate), Kyprolis® (carfilzomib), Velcade® (bortezomib), Casodex (bicalutamide), Iressa® (gefitinib), Venclexta™ (venetoclax) and Adriamycin™, (docorubicin) as well as a host of chemotherapeutic agents. Non-limiting examples of chemotherapeutic agents include alkylating agents such as thiotepa and cyclosphosphamide (Cytoxan™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, chlorocyclophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel and docetaxel; retinoic acid; esperamicins; capecitabine; and pharmaceutically acceptable salts, acids or derivatives of any of the above.

Also included as suitable chemotherapeutic cell conditioners are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO).

In certain embodiments, compounds and compositions of the present disclosure may be administered in combination with radiation therapy. Techniques for administering radiation therapy are known in the art, and these techniques can be used in the method described herein.

Radiation therapy may be administered before, during, and/or after treatment with compositions of the present disclosure.

The inhibitory activity of 2-bromo-2-nitro-1,3-propanediol (BP) can be reversed by administration of reducing agents such as, but not limited to, vitamin C and glutathione.

Because both cancerous (target cells) and noncancerous cells (non-target cells) use the thioredoxin system, administration of BP can affect non-target cells in addition to target cells. Beneficially, administration of above mentioned reducing agent can reverse the effect of BP and can help recover functionality and health of non-target cells, even if the non-target cells sustained damage. In certain embodiments, methods of the present disclosure further comprise administering to the subject reducing agents such as vitamin C or glutathione. In certain embodiments, reducing agents are administered before, during, and/or after treatment with compositions of the present disclosure.

In certain embodiments, the composition comprises at least one pharmaceutically acceptable excipient. In certain embodiments, the pharmaceutically acceptable excipient may be a carrier, adjuvant, diluent, buffer, stabilizer, preservative, lubricant, and/or the like. The pharmaceutically acceptable excipient(s) may be selected based on a number of factors, including intended bioavailability of the composition, the disease, disorder or condition being treated with the composition, the species of subject, the age, size and general condition of the subject, and the route of administration. Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, N.Y., USA); Remington's Pharmaceutical Sciences, 20th edition, pub. Lippincott, Williams & Wilkins, 2000 or Handbook of Pharmaceutical Excipients, 2nd edition, 1994.

Supplementary active ingredients also can be incorporated into the compositions. In certain embodiments, the formulation may further comprise additional ingredients, such as, for example, corn syrup solids, high-oleic safflower oil, coconut oil, soy oil, L-leucine, calcium phosphate tribasic, L-tyrosine, L-proline, L-lysine acetate, DATEM (an emulsifier), L-glutamine, L-valine, potassium phosphate dibasic, L-isoleucine, L-arginine, L-alanine, glycine, L-asparagine monohydrate, L-serine, potassium citrate, L-threonine, sodium citrate, magnesium chloride, L-histidine, L-methionine, ascorbic acid, calcium carbonate, L-glutamic acid, L-cystine dihydrochloride, L-tryptophan, L-aspartic acid, choline chloride, taurine, m-inositol, ferrous sulfate, ascorbyl palmitate, zinc sulfate, L-carnitine, alpha-tocopheryl acetate, sodium chloride, niacinamide, mixed tocopherols, calcium pantothenate, cupric sulfate, thiamine chloride hydrochloride, vitamin A palmitate, manganese sulfate, riboflavin, pyridoxine hydrochloride, folic acid, beta-carotene, potassium iodide, phylloquinone, biotin, sodium selenate, chromium chloride, sodium molybdate, vitamin D3 and/or cyanocobalamin.

Compositions of the present disclosure may be formulated according to methods known in the art. Proper formulation is dependent upon the route of administration chosen. Suitable routes of administration include, but are not limited to, oral, parenteral (e.g., intravenous, intraarterial, rectal, subcutaneous, intramuscular, intraorbital, intracapsular, intraspinal, intraperitoneal, or intrasternal), topical (transdermal, intranasal, ocular, buccal, and sublingual), intravesical, intrathecal, enteral, pulmonary, intralymphatic, intracavital, vaginal, transurethral, intradermal, aural, intramammary, orthotopic, intratracheal, intralesional, percutaneous, endoscopical, transmucosal, and intestinal administration.

The formulations may conveniently be presented in unit dosage form. Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid).

Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g., transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active compound.

Formulations suitable for administration by inhalation include those presented as an aerosol spray from a pressurized pack, with the use of a suitable propellant, such as dichlorodifluoromethane, trichlorofluoromethane, dichloro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas.

Examples of such dermal penetration enhancers include dimethylsulfoxide and related analogues.

When formulated as a topical emulsion, the oily phase may optionally comprise merely an emulsifier (otherwise known as an emulgent), or it may comprise a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilizers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate. Formulations suitable for vaginal administration may be presented as pessaries, tampons, creams, gels, pastes, foams or spray formulations containing in addition to the active compound, such carriers as are known in the art to be appropriate.

Compositions of the present disclosure may also be preferably formulated for parenteral administration, e.g., formulated for injection via intravenous, intraarterial, subcutaneous, rectal, subcutaneous, intramuscular, intraorbital, intracapsular, intraspinal, intraperitoneal, or intrasternal routes. The compounds of the disclosure for parenteral administration comprise an effective amount of the novel compounds in a pharmaceutically acceptable carrier. Dosage forms suitable for parenteral administration include solutions, suspensions, dispersions, emulsions or any other dosage form which can be administered parenterally. Techniques and compositions for making parenteral dosage forms are known in the art.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilizers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 g/ml, for example from about 10 ng/ml to about 1 g/ml. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use.

Extemporaneous injection solutions and suspensions may be prepared from sterile powders, granules, and tablets. Formulations may be in the form of liposomes or other microparticulate systems which are designed to target the active compound to blood components or one or more organs.

All publications and patent applications in this specification are indicative of the level of ordinary skill in the art to which this disclosure pertains. All publications and patent applications are herein incorporated by reference to the same extent as if each individual publication or patent application was specifically and individually indicated as incorporated by reference.

### EXAMPLES

### Example 1

This example describes the materials and methods used in Example 2.

HeLa cells were purchased from the American Type Culture Collection. Cells were grown in DMEM medium supplemented with 10% fetal calf serum and penicillin/streptomycin (100 μg/ml), in humid atmosphere of 5% CO2 at 37° C. 2-Bromo-2-nitro-1,3-propanediol (BP) was purchased from Thermo Scientific (USA). Rat liver thioredoxin reductase (TR) was purchased from SIGMA (St Louis MO, USA) and human recombinant thioredoxin reductase was purchased from Invitrogen. Illustra MicroSpin Sephadex™ G-25 columns containing DNA grade F resin was purchased from GE Healthcare.

### Inhibition of TR Activity

BP stock solutions were prepared by dissolving the compound in PBS to achieve the desired concentrations. HeLa cell lysate was prepared by homogenizing cell pellet in cold homogenizing buffer (50 mM potassium phosphate, pH 7.4 containing 1 mM EDTA) and then centrifuging at 10,000×g for 15 minutes at 4° C. and used the supernatant for TR activity measurements. TR activity in recombinant human TR (15 μg) and Hela cell lysate (100) μg total protein) was measured using Cayman's (Cayman Chemicals, Ann Arbor MI, USA) TR assay kit following manufacturer's instructions. TR activity was calculated using the formula given in the instruction manual. To detect the TR inhibitory activity of BP, the desired concentrations of the compound was added to TR reaction mixture and TR activity was measured.

### Reactivation of BP Inactivated TR by DTT

Human recombinant TR or HeLa cell lysates were treated with BP to get a final BP concentration of 25 μM and incubated at room temperature for 5 minutes. Then the reaction mixture was divided into two aliquots and one aliquot was used to measure TR activity and other aliquot was treated with 100 μM dithiothreitol (DTT) for 10 minutes. After incubation period the mixture was passed through a Sephadex™ G-25 column to remove excess DTT and BP. DTT and BP removed mixture was then used to measure TR activity.

### Cytotoxic Effects of BP

HeLa cells were cultured and grown to approximately 70%-80% confluency in DMEM medium supplemented with fetal calf serum and antibiotics. To investigate the cytotoxic effects BP, the HeLa cells were treated with various concentrations of the compound. A stock solution of BP was prepared by dissolving the compound in PBS to achieve the desired concentrations. The treated cells were incubated with BP for two different time points, ensuring exposure to the compound for sufficient durations to observe potential cytotoxic effects. The specific time points chosen were 24 and 48 hours. To assess cell viability, the MTT (3-(4,5-dimethylthiazol-2-yl)-2,5-diphenyltetrazolium bromide) assay was employed. In brief, after the treatment period, the culture medium was removed, and the cells were washed with PBS to remove any residual compound. Subsequently, the MTT reagent, which is converted to a colored formazan product by metabolically active cells, was added to each well containing the treated cells. The cells were then incubated for a specific duration to allow the conversion of MTT into formazan crystals. Following this incubation period, the formazan crystals were solubilized, and the absorbance at 570 nm was measured spectrophotometrically using a microplate reader. The absorbance values obtained from the MTT assay were used to determine the relative cell viability of the treated HeLa cells compared to control cells.

### Effect of BP on Induction of Apoptosis

HeLa cells were seeded and allowed to reach 70%-80% confluency. The cells were then treated with a concentration of 25 μM BP for a duration of 24 hours. This concentration and time point were determined based on preliminary experiments. Following the treatment period, both the treated and untreated cells were gently washed twice with PBS to remove any residual compound or culture media. The cells were then fixed by adding 4% paraformaldehyde solution to the culture dishes and incubating for 10 minutes at room temperature. After fixation, the cells were washed three times with PBS to remove excess paraformaldehyde. To assess apoptosis, the fixed cells were stained with Hoechst 33342 reagent, a fluorescent DNA-binding dye commonly used to visualize nuclear morphology changes associated with apoptosis. The Hoechst 33342 reagent was added to the culture dishes containing the fixed cells and incubated for 10 minutes at room temperature. Subsequently, the cells were washed three times with PBS to remove any unbound dye. Finally, the stained cells were observed and photographed using a ZEISS fluorescence microscope (Carl Zeiss Microscopy GmbH, Jena/Germany) to detect apoptotic morphological changes such as nuclear condensation and fragmentation.

Detection of c-Fos mRNA

RNA was extracted from BP treated and untreated HeLa cells using GeneJet RNA purification kit (Thermo Scientific, USA). The nucleotide sequences for primers and probe c-fos were purchased from Integrated DNA Technologies (Coralville, IA). cDNA was synthesized using iScript™ Reverse Transcription Supermix for RT-qPCR (Bio-Rad, Hercules, CA). RT-ddPCR was performed on the QX200 Droplet Digital PCR system (Bio-Rad, Hercules, CA) using the ddPCR™ Kit for probes (Bio-Rad).

### Example 2

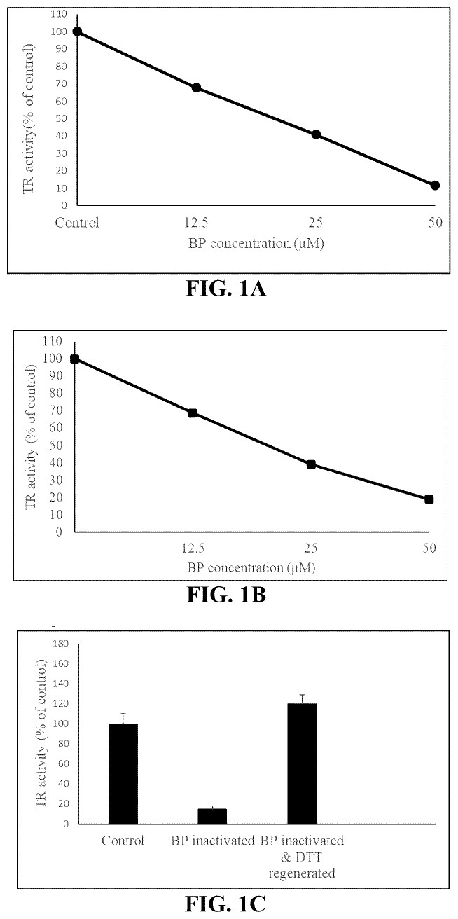

The activity of human recombinant TR was determined in the presence or absence of different concentrations of BP (12.5, 25, and 50 μM), as outlined in Example 1. The treatment of TR with 12.5, 25, and 50 μM BP resulted in reductions of activity by 32%, 59.25%, and 88.4%, respectively (see FIG. 1A). These findings suggest a potent inhibitory effect of BP on TR. The IC50 value for human recombinant TR was calculated to be 21 μM. Furthermore, we investigated the ability of BP to inhibit TR activity in a cell lysate obtained from the HeLa human cervical cancer cell line. The TR activities of both treated and untreated cell lysates (200 μg total protein) were measured using the previously described method. Treatment with 12.5, 25, and 50 μM BP led to reductions in TR activity by 31%, 60.8%, and 81%, respectively (see FIG. 1B). The IC50 value for the HeLa cell lysate was determined to be 20.5 μM. We investigated to determine whether the inhibition of TR mediated by BP is reversible or irreversible. In FIG. 1C, it is shown that when human recombinant TR was treated with 50 μM BP, there was an 85% inhibition of TR activity. However, when the BP-inactivated TR was treated with reducing agent DTT at room temperature for 10 minutes, followed by the removal of DTT using a Sephadex™ G-25 column, complete recovery of TR activity was observed. This indicates that the BP-mediated TR inhibition is reversible, as demonstrated in FIG. 1C.

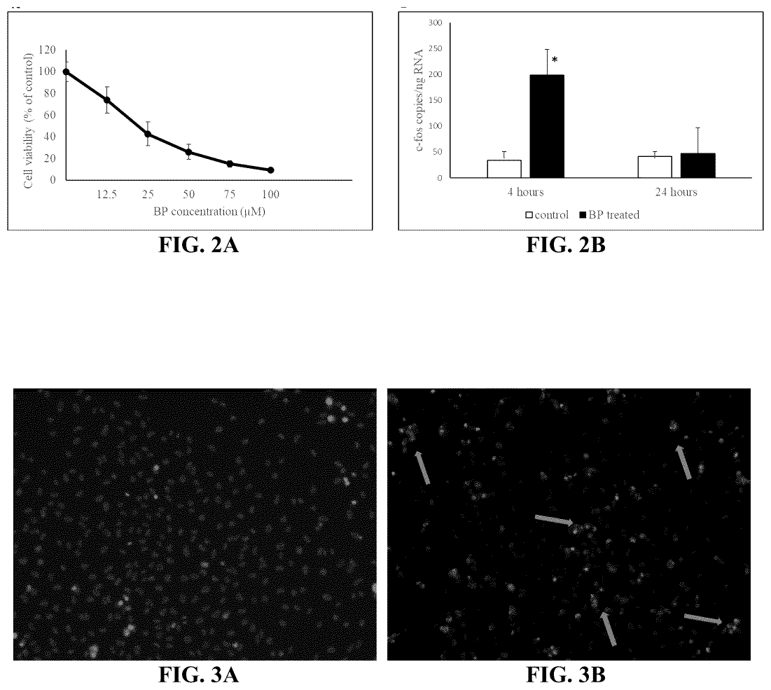

To further investigate the impact of TR inhibition by BP on the viability of HeLa cells, we treated the cells with four different concentrations of BP (12.5, 25, 50, and 100 μM) for a duration of 24 hours. Cell viability was assessed using the MTT assay, as described in Example 1. The results, presented in FIG. 2A, indicate that the inhibitory effect on cell viability increased in a dose-dependent manner. Specifically, BP concentrations of 12.5, 25, 50, and 100 μM resulted in a decrease in cell viability by 26%, 57%, 74%, 85%, and 91%, respectively. By calculating the LD50 for BP, we determined it to be 21 μM. This experiment shows us that BP has the potential to induce cell death. Cell death may take place either via apoptosis or necrosis. To know which pathway is responsible we have studied c-fos mRNA expression in BP treated or untreated HeLa cells. Cells were either treated or untreated with BP (50 μM) for 4 or 24 hours and c-fos mRNA copy number determined using droplet digital PCR. As shown in FIG. 2B, there is a statistically significant transient increase in c-fos mRNA level at 4 hours after BP treatment and return to normal levels after 24 hours. The effect of BP on cell apoptosis was investigated by treating cells with BP and comparing them to untreated cells. To assess apoptotic changes, both the BP-treated and untreated cells were stained with Hoechst 33342 and observed under a fluorescence microscope. FIG. 3A shows untreated HeLa cells stained with Hoechst 33342. Those untreated cells showed no nuclear condensation or fragmentation. However, observation of the BP-treated cells (FIG. 3B) revealed distinct nuclear fragmentation and condensation when compared to the untreated cells. These apoptotic markers, characterized by fragmented and condensed nuclei, strongly indicate that BP induces cell apoptosis. These findings demonstrate that BP treatment significantly influences the apoptotic process, as evidenced by the pronounced morphological changes observed in the treated cells. The observed nuclear fragmentation and condensation suggest that BP has a potential pro-apoptotic effect on the treated cells.

### DISCUSSION

The thioredoxin system (TRXS) is a very important redox regulator in mammalian cells. It protects cells from oxidative stress by maintaining the balance of the thiol-disulfide redox status. Therefore, changes in TRXS expression level may change cellular physiology.

There are numerus reports that show TRXS is overexpressed in cancer cells. Without being limited by theory, elevated levels of TRXS could disrupt cell cycle regulation through excessive activation of growth factors and inhibition of apoptosis, leading to the malignant transformation of cells. Hence TRXS has emerged as a potential molecular target for cancer therapy.

Both TRX and TR have critical thiol groups in their active sites and oxidation of these thiol groups make TR and TRX inactive. BP is a well-known antimicrobial agent widely use in pharmaceutical and cosmetic industries as a preservative. This compound has the potential to oxidize protein thiol groups. Hence, this study was initiated to evaluate its potential as a TR inhibitor and an anticancer agent.

The results obtained from this study demonstrate the potent inhibitory effect of BP on the activity of human recombinant TR. A dose-dependent reduction was observed in TR activity upon treatment with increasing concentrations of BP, both in the purified enzyme and in the cell lysate obtained from HeLa cells. In the purified enzyme experiments, the activity of human recombinant TR was significantly reduced by 32%, 59.25%, and 88.4% upon treatment with 12.5, 25, and 50 μM BP, respectively. These findings indicate that BP is a strong inhibitor of TR activity. The calculated IC50 value of 21 μM suggests that BP is effective at low concentrations in inhibiting TR.

Furthermore, the effect of BP on TR activity in a cellular context was investigated using HeLa cell lysates. Similar to the purified enzyme experiments, treatment with BP led to a dose-dependent reduction in TR activity in the cell lysate. Specifically, TR activity was reduced by 31%, 60.8%, and 81% upon treatment with 12.5, 25, and 50 μM BP, respectively. The IC50 value of 20.5 μM indicates that BP exhibits comparable inhibitory potency in the cellular context.

To gain further insight into the nature of BP-mediated TR inhibition, its reversibility was assessed. Results demonstrated that the inhibition of TR by BP is reversible. Treatment of BP-inactivated TR with reducing agent DTT resulted in complete recovery of TR activity. This suggests that the oxidation of thiol groups on TR is not irreversible and that the inhibitory effect can be reversed, potentially offering a promising avenue for intervention strategies.

The impact of TR inhibition by BP on the viability of HeLa cells was also investigated. The results revealed a dose-dependent decrease in cell viability upon BP treatment. Increasing concentrations of BP (12.5, 25, 50, and 100 μM) led to a corresponding decrease in cell viability by 26%, 57%, 74%, 85%, and 91%, respectively. The LD50 value of 21 μM indicates that BP is effective in inducing cell death in HeLa cells.

To elucidate the mechanism of cell death induced by BP, the pathways of apoptosis and necrosis were explored. Transient induction of c-fos is implicated in the activation apoptosis in various cell types. Studies have shown that c-fos induction is necessary before the apoptotic chromatin condensation and oligonucleosomal DNA fragmentation. The instant findings suggest that BP-induced cell death occurs primarily through apoptosis rather than necrosis. A transient increase was observed in c-fos mRNA expression, a marker of cellular stress and apoptosis, at 4 hours following BP treatment. However, the c-fos mRNA levels returned to normal after 24 hours, indicating a transient apoptotic response. This temporal pattern suggests that BP treatment triggers an early apoptotic event, which subsides over time.

The morphological changes in HeLa cells upon treatment were examined to further confirm the apoptotic effect of BP. BP-treated cells exhibited distinct nuclear fragmentation and condensation, as evidenced by Hoechst 33342 staining. In contrast, untreated cells showed no such changes. The presence of fragmented and condensed nuclei strongly supports the notion that BP induces apoptosis in HeLa cells.

In summary, the instant study demonstrates that BP is a potent inhibitor of TR activity, both in purified enzyme and cellular contexts. The reversible nature of BP-mediated TR inhibition provides potential therapeutic opportunities. Moreover, our findings indicate that BP induces cell death primarily through the apoptotic pathway, as evidenced by changes in c-fos mRNA expression and morphological features of apoptosis. Due to the TR inhibitory effect and the pro-apoptotic properties of BP, BP offers a novel anticancer therapy.

What is claimed is:

1. A method of treating cancer in a subject in need thereof, comprising:
administering to the subject a composition comprising a therapeutically effective amount of 2-bromo-2-nitro-1,3-propanediol.

2. The method of claim 1, wherein the cancer is thyroid cancer, lung cancer, gastric cancer, colorectal cancer, hepatic cancer, urogenital cancer, brain cancer, breast cancer, pancreatic cancer, or hematologic cancer.

3. The method of claim 2, wherein the cancer is metastatic cancer.

4. The method of claim 2, wherein the composition is administered orally, intravenously, or intramuscularly.

5. The method of claim 4, wherein the composition is administered in combination with a chemotherapeutic agent.

6. The method of claim 4, wherein the composition is administered in combination with radiation therapy.

7. The method of claim 4, wherein the composition further comprises a pharmaceutically acceptable excipient.

8. The method of claim 7, wherein the pharmaceutically acceptable excipient comprises a carrier, adjuvant, diluent, buffer, stabilizer, preservative, and/or lubricant.

9. The method of claim 2, wherein the subject is human.

10. The method of claim 1, wherein the method further comprises administering to the subject vitamin C and/or glutathione.

11. A method of inhibiting the activity of thioredoxin reductase in a cell of a subject, comprising:
administering to the subject a composition comprising 2-bromo-2-nitro-1,3-propanediol.

12. The method of claim 11, wherein the subject has cancer.

13. The method of claim 12, wherein the cancer is thyroid cancer, lung cancer, gastric cancer, colorectal cancer, hepatic cancer, urogenital cancer, brain cancer, breast cancer, or hematologic cancer.

14. The method of claim 13, wherein the cancer is metastatic cancer.

15. The method of claim 13, wherein the composition is administered orally, intravenously, or intramuscularly.

16. The method of claim 15, wherein the composition is administered in combination with a chemotherapeutic agent.

17. The method of claim 15, wherein the composition is administered in combination with radiation therapy.

18. The method of claim 15, wherein the composition further comprises a pharmaceutically acceptable excipient.

19. The method of claim 18, wherein the pharmaceutically acceptable excipient comprises a carrier, adjuvant, diluent, buffer, stabilizer, preservative, and/or lubricant.

20. The method of claim 11, wherein the method further comprises administering to the subject vitamin C and/or glutathione to restore the activity of thioredoxin reductase.
